(19) United States
(12) Patent Application Publication
(10) Pub. No.: US 2026/0365756 A1
(43) Pub. Date: Nov. 19, 2026

(54) ANTIBODY-CONTAINING PREPARATION

(71) Applicant: Chugai Seiyaku Kabushiki Kaisha, Tokyo (JP)

(72) Inventors: Atsushi Saeki, Tokyo (JP); Shaw Nishizawa, Tokyo (JP); Hitoshi Sasaki, Tokyo (JP); Chifumi Imai, Tokyo (JP); Tomoyuki Igawa, Shizuoka (JP)

(73) Assignee: Chugai Seiyaku Kabushiki Kaisha, Tokyo (JP)

(21) Appl. No.: 19/353,963

(22) Filed: Oct. 9, 2025

Related U.S. Application Data
(63) Continuation of application No. 16/093,495, filed on Apr. 27, 2017, now Pat. No. 12,460,014.

(30) Foreign Application Priority Data
Apr. 28, 2016 (JP) 2016-090590

(51) Int. Cl. C07K 16/36; A61K 9/08; A61K 47/18; A61K 47/34

(52) U.S. Cl. CPC C07K 16/36; A61K 9/08; A61K 47/183; A61K 47/34; C07K 2317/31; C07K 2317/51; C07K 2317/515; C07K 2317/565

(57) ABSTRACT

The present invention relates to stable antibody-containing solution formulations in which aggregate formation of Emicizumab (ACE910) which is a bispecific antibody functionally substituting for FVIII, is suppressed. Specifically, the present invention relates to the above-mentioned antibody-containing solution formulations of pH 4.5 to 6.5 that contain the aforementioned bispecific antibody at 20 to 180 mg/mL, 10 mM to 40 mM histidine-aspartate buffer, Poloxamer 188 at 0.2 to 1 mg/mL, and 100 mM to 300 mM arginine.

### CROSS-REFERENCE TO RELATED APPLICATIONS

This application is a continuation application of U.S. application Ser. No. 16/093,495, filed Oct. 12, 2018, which is the National Stage of International Application No. PCT/JP2017/016658, filed on Apr. 27, 2017, which claims the benefit of Japanese Application Serial No. 2016-090590, filed on Apr. 28, 2016. The entire contents of application Ser. No. 16/093,495 are hereby incorporated by reference.

### SEQUENCE LISTING

This application contains a Sequence Listing that has been submitted electronically as an XML file named 14875-0265002_SL_ST26.xml. The XML file, created on Sep. 25, 2025, is 21,262 bytes in size. The material in the XML file is hereby incorporated by reference in its entirety.

### TECHNICAL FIELD

The present invention relates to formulations comprising a bispecific antibody functionally substituting for blood coagulation factor VIII (FVIII) that binds to blood coagulation factor IX (FIX) and/or activated blood coagulation factor IX (FIXa) and blood coagulation factor X (FX).

### BACKGROUND ART

Bispecific antibodies functionally substituting for FVIII that bind to blood coagulation factor IX (FIX) and/or activated blood coagulation factor IX (FIXa) and blood coagulation factor X (FX) have been discovered (Non-Patent Literature 1 and 2; Patent Literature 1 to 3). The bispecific antibody Emicizumab (ACE910) ameliorates the decrease in coagulation reaction due to FVIII deficiency and dysfunction by functionally substituting for FVIII; therefore, clinical trials are being conducted on hemophilia A patients.

Many solution formulations of antibodies have been developed, and solution formulations of high-concentrated antibodies reported so far are formulations using histidine and arginine (Patent Literature 4) and formulations using a histidine/aspartate buffer (Patent Literature 5). Meanwhile, a stable liquid pharmaceutical antibody formulation comprising amyloid β (Aβ) that uses histidine/histidine-HCl as the buffer (Patent Literature 6) has been reported.

However, stable solution formulations in which aggregate formation and/or components with charge heterogeneity are suppressed have not been reported for solution formulations comprising the aforementioned bispecific antibodies.

### CITATION LIST

### Patent Literature

[Patent Literature 1] WO2005/035756
[Patent Literature 2] WO2006/109592
[Patent Literature 3] WO2012/067176
[Patent Literature 4] WO2002/030463
[Patent Literature 5] WO2011/090088
[Patent Literature 6] WO2013/131866

### Non-Patent Literature

[Non-Patent Literature 1] Nat Med. 2012; 18 (10): 1570-74
[Non-Patent Literature 2] PLOS One. 2013; 8 (2): e57479

### SUMMARY OF THE INVENTION

### Problems to be Solved

An objective of the present invention is to provide stable solution formulations comprising Emicizumab (ACE910) which is a bispecific antibody functionally substituting for FVIII that binds to FIX and/or FIXa and FX.

### Means for Solving the Problems

As a result of dedicated research to accomplish the above-mentioned objective, the present inventors discovered that a solution formulation of pH 4.5 to 6.5 that comprises the aforementioned bispecific antibody at 20 to 180 mg/mL, 10 mM to 40 mM histidine/aspartate buffer, Poloxamer 188 at 0.2 to 1 mg/mL, and 100 mM to 300 mM arginine, can be a stable antibody-containing solution formulation in which aggregate formation and/or components with charge heterogeneity are suppressed, and thereby completed the present invention.

Specifically, the present invention provides the following:
[1] An antibody solution formulation of pH 4.5 to 6.5, which comprises:
   a bispecific antibody at 20 to 180 mg/mL, wherein a first polypeptide and a third polypeptide form a pair, and a second polypeptide and a fourth polypeptide form a pair, wherein the first polypeptide comprises an H chain comprising the amino acid sequences of H-chain CDRs 1, 2, and 3 of SEQ ID NOs: 1, 2, and 3 (H-chain CDRs of Q499), respectively; the second polypeptide comprises an H chain comprising the amino acid sequences of H-chain CDRs 1, 2, and 3 of SEQ ID NOs: 4, 5, and 6 (H-chain CDRs of J327), respectively; and the third polypeptide and the fourth polypeptide comprise a common L chain comprising the amino acid sequences of L-chain CDRs 1, 2, and 3 of SEQ ID NOs: 7, 8, and 9 (L-chain CDRs of L404), respectively;
   10 mM to 40 mM histidine/aspartate buffer;
   0.2 to 1 mg/mL Poloxamer 188; and
   100 mM to 300 mM arginine.
[2] The antibody solution formulation of [1], wherein in the bispecific antibody, the first polypeptide and the third polypeptide form a pair, and the second polypeptide and the fourth polypeptide form a pair, wherein the first polypeptide comprises an H chain comprising the amino acid sequence of SEQ ID NO: 10; the second polypeptide comprises an H chain comprising the amino acid sequence of SEQ ID NO: 11, and the third polypeptide and the fourth polypeptide comprise a common L chain of SEQ ID NO: 12.
[3] The antibody solution formulation of [1] or [2], wherein the concentration of Poloxamer 188 is 0.5 mg/mL.
[4] The antibody solution formulation of any one of [1] to [3], wherein said pHis 6.0.
[5] The antibody solution formulation of any one of [1] to [4], wherein the concentration of histidine/aspartate buffer is 20 mM.
[6] The antibody solution formulation of any one of [1] to [5], wherein the concentration of arginine is 150 mM.
[7] The antibody solution formulation of any one of [1] to [6], which does not substantially contain a chloride ion or an acetate ion.
[8] An antibody solution formulation of pH 6, which comprises:
   a bispecific antibody at 20 to 180 mg/mL, wherein a first polypeptide and a third polypeptide form a pair, and a second polypeptide and a fourth polypeptide form a pair, wherein the first polypeptide comprises an H chain comprising the amino acid sequence of SEQ ID NO: 10; the second polypeptide comprises an H chain comprising the amino acid sequence of SEQ ID NO: 11; and the third polypeptide and the fourth polypeptide comprise a common L chain of SEQ ID NO: 12;
   20 mM L-histidine/aspartate buffer;
   0.5 mg/mL Poloxamer 188; and
   150 mM L-arginine.
[9] The antibody solution formulation of any one of [1] to [8], for use in subcutaneous administration.
[10] The antibody solution formulation of any one of [1] to [9], for use in the treatment of hemophilia A.
[11] A method for stabilizing an antibody in an antibody-containing solution formulation, which comprises adding a histidine/aspartate buffer, Poloxamer 188, and arginine to the solution, whereby the concentration of the histidine/aspartate buffer is 10 mM to 40 mM, the concentration of Poloxamer 188 is 0.2 to 1 mg/mL, and the concentration of arginine is 100 mM to 300 mM.
[12] A method for suppressing the association (aggregate formation) of an antibody in an antibody-containing solution formulation, which comprises adding a histidine/aspartate buffer, Poloxamer 188, and arginine to the solution, whereby the concentration of the histidine/aspartate buffer is 10 mM to 40 mM, the concentration of Poloxamer 188 is 0.2 to 1 mg/mL, and the concentration of arginine is 100 mM to 300 mM.
[13] A method for suppressing a component with charge heterogeneity in an antibody-containing formulation, which comprises adding a histidine/aspartate buffer to the solution, wherein the concentration of histidine/aspartate buffer is 10 mM to 40 mM.

### Effect of the Invention

The present invention provides antibody-containing formulations which show excellent stability. Furthermore, providing antibody-containing formulations in which aggregate formation and/or components with charge heterogeneity are suppressed in its solution state has been also enabled by the present invention.

### BRIEF DESCRIPTION OF THE DRAWINGS

FIG. 1 shows photographs indicating insoluble foreign substances present after the shaking tests of EXAMPLE 8 (a: 0 mg/mL Poloxamer188; b: 0.5 mg/mL Poloxamer188).

FIG. 2 shows graphs indicating the number of insoluble microparticles (microparticles/mL) present after the shaking tests and freeze-thawing of EXAMPLE 8.

### MEANS FOR CARRYING OUT THE INVENTION

The present invention will be described in detail below.

The present invention provides a solution formulation of pH 4.5 to 7.5 that comprises: Emicizumab (ACE910) at 20 to 180 mg/mL which is a bispecific antibody functionally substituting for FVIII that binds to FIX and/or FIXa and FX; 10 mM to 40 mM histidine/aspartate buffer; Poloxamer 188 at 0.2 to 1 mg/mL; and 100 mM to 300 mM arginine.

Emicizumab (ACE910), which is the aforementioned bispecific antibody, is described below.

A bispecific antibody (Q499-z121/J327-z119/L404-k) where a first polypeptide and a third polypeptide form a pair, and a second polypeptide and a fourth polypeptide form a pair; where the first polypeptide comprises an H chain comprising the amino acid sequences of H-chain CDRs 1, 2, and 3 of SEQ ID NOs: 1, 2, and 3 (H-chain CDRs of Q499), respectively; the second polypeptide comprises an H chain comprising the amino acid sequences of H-chain CDRs 1, 2, and 3 of SEQ ID NOs: 4, 5, and 6 (H-chain CDRs of J327), respectively; and the third polypeptide and the fourth polypeptide comprise a common L chain comprising the amino acid sequences of L-chain CDRs 1, 2, and 3 of SEQ ID NOs: 7, 8, and 9 (L-chain CDRs of L404), respectively.

More specifically, the aforementioned bispecific antibody is a bispecific antibody where a first polypeptide and a third polypeptide form a pair, and a second polypeptide and a fourth polypeptide form a pair; where the first polypeptide comprises an H chain comprising the amino acid sequence of H-chain variable region of SEQ ID NO: 13, the second polypeptide comprises an H chain comprising the amino acid sequence of H-chain variable region of SEQ ID NO: 14, and the third polypeptide and the fourth polypeptide comprise a common L chain comprising the amino acid sequence of L-chain variable region of SEQ ID NO: 15.

More specifically, the aforementioned bispecific antibody is a bispecific antibody (Q499-z121/J327-z119/L404-k) where a first polypeptide and a third polypeptide form a pair, and a second polypeptide and a fourth polypeptide form a pair; where the first polypeptide comprises an H chain comprising the amino acid sequence of SEQ ID NO: 10, the second polypeptide comprises an H chain comprising the amino acid sequence of SEQ ID NO: 11, and the third polypeptide and the fourth polypeptide comprise a common L chain of SEQ ID NO: 12. Such antibodies can be obtained by the methods described in WO2005/035756, WO2006/109592, WO2012/067176, and such.

The antibody concentration in a formulation of the present invention is not particularly limited, but is preferably 20 mg/mL to 180 mg/mL. Examples include 20 mg/mL, 30 mg/mL, 40 mg/mL, 120 mg/mL, 150 mg/mL, and 180 mg/mL. The upper limit of the antibody concentration of a formulation of the present invention is not particularly limited, but is ordinarily 250 mg/mL.

Antibodies used in the present invention are not particularly limited so long as they bind to a desired antigen, and they may be polyclonal or monoclonal antibodies. Monoclonal antibodies are preferred in that homogeneous antibodies can be stably produced.

Amino acids contained in the amino acid sequences of the present invention may be post-translationally modified (for example, the modification of an N-terminal glutamine into a pyroglutamic acid by pyroglutamylation is well-known to those skilled in the art). Naturally, such post-translationally modified amino acids are included in the antibodies used in the present invention.

In the present invention, the phrase “functionally substituting for FVIII” means recognizing FIX or FIXa, and FX, and promoting FX activation by FIXa (promoting FXa production by FIXa). FXa production-promoting activity can be evaluated using, for example, a measurement system comprising FXIa, FX, synthetic substrate S-2222 (synthetic substrate of FXa), and phospholipids. Such a measurement system shows a correlation with the disease severity and clinical symptoms in hemophilia A cases (Rosen S, Andersson M, Blomba\ddot{ } ck M et al. Clinical applications of a chromogenic substrate method for determination of FVIII activity. Thromb Haemost 1985; 54:811-23).

In the present invention, the term “common L chain” refers to an L chain that can form pairs with each of two or more different H chains, and show binding ability to each antigen. Herein, the term “different H chains” preferably refers to H chains of antibodies against different antigens, but is not limited thereto; it refers to H chains whose amino acid sequences are different from each other. Common L chains can be obtained, for example, according to the method described in WO 2006/109592.

In the present invention, the term “stable antibody-containing formulation” refers to a formulation in which aggregates and/or components with charge heterogeneity from proteins such as antibodies are difficult to be generated, i.e., the formulations in which deterioration reactions, including generation of insoluble aggregates, soluble aggregates, components with charge heterogeneity, are difficult to occur in the solution.

“Components with charge heterogeneity” refer to components having protein surface charges that are different from those of the major component due to deamidation, oxidation, hydrolysis, and such.

In the present invention, “polypeptide” generally refers to peptides and proteins having a length of approximately ten amino acids or longer. Ordinarily, they are biologically derived polypeptides, but are not particularly limited thereto, and may be, for example, polypeptides comprising an artificially designed sequence. Furthermore, they may be any naturally-occurring polypeptides, synthetic polypeptides, recombinant polypeptides, or such. Additionally, fragments of the above-mentioned polypeptides are also included in the polypeptides of the present invention.

The term “antibody” is used in the broadest sense, and includes monoclonal antibodies, polyclonal antibodies, dimers, multimers, multispecific antibodies (such as bispecific antibodies), antibody derivatives, and modified antibodies (Miller K et al. J Immunol. 2003, 170 (9), 4854-61) so long as they show a desired biological activity. The antibodies may be mouse antibodies, human antibodies, humanized antibodies, chimeric antibodies, or those derived from another species, or artificially synthesized antibodies. The antibodies disclosed herein can be of any type (for example, IgG, IgE, IgM, IgD, and IgA), class (for example, IgG1, IgG2, IgG3, IgG4, IgAQ1 and IgA2) or subclass of immunoglobulin molecules. The immunoglobulins can be derived from any species (for example, human, mouse, or rabbit). The terms “antibody”, “immune globulin” and “immunoglobulin” are used interchangeably in a broad sense.

“Bispecific antibody” refers to an antibody having two variable regions that each recognize different epitopes, where the variable regions are present in the same antibody molecule. Bispecific antibodies may be antibodies that recognize two or more different antigens, or antibodies that recognize two or more different epitopes on the same antigen. Bispecific antibodies may include not only whole antibodies but antibody derivatives.

Recombinant antibodies produced by using genetic engineering techniques can be used as the antibodies. A recombinant antibody can be obtained by cloning a DNA encoding the antibody from hybridomas or antibody-producing cells such as sensitized lymphocytes that produce antibodies; inserting this into a vector; and then introducing it into hosts (host cells) to produce the antibody.

Bispecific antibodies are not limited to those of the IgG type; for example, IgG-type bispecific antibodies can be secreted from a hybrid hybridoma (quadroma) produced by fusing two types of hybridomas that produce IgG antibodies (Milstein C. et al., Nature 1983, 305:537-540). They can also be secreted by introducing into cells the L-chain and H-chain genes constituting the two kinds of IgGs of interest, i.e., a total of four kinds of genes, to co-express the genes.

Antibodies of the present invention can be produced by methods known to those skilled in the art. Specifically, a DNA encoding the antibody of interest is inserted into an expression vector. The insertion into the expression vector is carried out such that the expression will take place under the control of expression regulatory regions such as an enhancer and a promoter. Next, host cells are transformed using this expression vector to express the antibody. Appropriate combinations of a host and an expression vector can be used in this case.

The antibodies of the present invention thus obtained can be isolated from the inside of host cells or the outside of the cells (medium, etc.), and purified to be substantially pure, homogeneous antibodies. The antibodies can be separated and purified by methods ordinarily used for separating and purifying antibodies, and the methods are not limited in any way. For example, the antibodies can be separated and purified by appropriately selecting and combining column chromatography, filtration, ultrafiltration, salting-out, solvent precipitation, solvent extraction, distillation, immunoprecipitation, SDS-polyacrylamide gel electrophoresis, isoelectrofocusing, dialysis, recrystallization, and such.

In a preferred aspect, the histidine/aspartate buffer in a formulation of the present invention is a buffer prepared by titrating a solution such as an aqueous solution supplemented with histidine as a free amino acid with a liquid such as an aqueous solution containing aspartic acid as a free amino acid. Alternatively, the buffer can be prepared by adding the amino acids in the reverse order, or by direct titration with powders.

The present inventors conducted freeze-thawing tests, thermal acceleration tests, long term storage tests, and cryopreservation tests to assess the effects of various additives on the stability of samples containing the above-mentioned bispecific antibodies during their storage. As a result, the present inventors discovered that aggregate formation and/or components with charge heterogeneity are suppressed by using a histidine buffer, as compared to phosphate buffer, citrate buffer, and acetate buffer.

Furthermore, the present inventors discovered that aggregate formation and/or components with charge heterogeneity are suppressed by using aspartic acid which is an acidic amino acid as a counter ion species for the buffer, i.e., by using histidine/aspartate buffer as the buffer.

The concentration (amount) of the histidine/aspartate buffer in formulations of the present invention is preferably 10 to 100 mM, and more preferably 10 to 40 mM. Furthermore, examples of the concentration (amount) of the histidine/aspartate buffer are 10 mM, 20 mM, and 40 mM.

Furthermore, compared to sodium chloride which is reported to be a stabilizer for antibody-containing formulations, addition of arginine was found to show higher stabilization effects (i.e., effects of suppressing aggregate formation and effects of suppressing components with charge heterogeneity).

The concentration (amount) of arginine in formulations of the present invention is preferably 100 mM to 300 mM. Examples of the arginine concentration (amount) include 100 mM, 150 mM, 200 mM, and 300 mM.

The solution pH of a formulation of the present invention is preferably 4.5 to 6.5, more preferably 5.5 to 6.5, and even more preferably 5.5 to 6. Examples of pH include 5.5 and 6.

Surfactants contained in formulations of the present invention are, for example polysorbate 20 (PS20), and Pluronic F-68 (Poloxamer 188: polyethylene (160) polyoxypropylene (30) glycol), and Poloxamer 188 is particularly preferred. The amount of Poloxamer 188 (or PX188) added to a formulation of the present invention is preferably 0.2 mg/mL to 1 mg/mL. Examples of the amount of Poloxamer 188 added to the formulation include 0.2 mg/mL, 0.5 mg/mL, 0.8 mg/mL, and 1 mg/mL.

The histidine used in the present invention may be histidine itself or a derivative thereof, and L-histidine is particularly desirable. The arginine used in the present invention may be arginine itself, a derivative thereof, or a salt thereof, and L-arginine or a salt thereof is particularly desirable. Preferred salts of arginine include aspartate salt and glutamate salt.

The formulations of the present invention may further contain amino acids. Preferred amino acids for use in the present invention are natural amino acids or amino acid derivatives, and particularly preferred amino acids are L-methionine and L-proline.

The formulations of the present invention may further contain sugars. Preferred sugars used in the present invention are sucrose, trehalose, meglumine, and sorbitol.

The amount of amino acid or sugar added to the formulations of the present invention is generally 1 mM to 1000 mM, preferably 5 mM to 500 mM, and more preferably 10 mM to 300 mM.

The formulations of the present invention may further contain inorganic salts. The preferred inorganic salts used in the present invention are magnesium salts and calcium salts.

Furthermore, it is preferred that the formulations of the present invention do not contain anions other than aspartic acid as a counter ion for the buffer (buffering agent) or stabilizer. In an aspect, examples of such formulations include formulations that do not substantially contain chloride ion or acetate ion. “Substantially do not contain chloride ion or acetate ion” means that the concentrations of chloride ion and acetate ion are, for example, 5 mM or less, preferably 2 mM or less, and more preferably 1 mM or less. Highly stable antibody-containing formulations can be produced without increasing the osmotic pressure by using aspartic acid which has a large stabilizing effect as a counter ion and not substantially including chloride ion or acetate ion with a small stabilization effect.

If needed, the formulations of the present invention may additionally contain appropriate cryoprotectants, suspending agents, solubilizing agents, isotonizing agents, preservatives, adsorption inhibitors, diluents, excipients, pH adjustors, analgesics, sulfur-containing reducing agents, antioxidants, and such.

Cryoprotectants include, for example, sugars such as trehalose, sucrose, and sorbitol.

Solubilizing agents include, for example, polyoxyethylene hardened castor oil, polysorbate 80, nicotinamide, polyoxyethylene sorbitan monolaurate, macrogol, and castor oil fatty acid ethyl ester.

Isotonizing agents include, for example, sodium chloride, potassium chloride, and calcium chloride.

Preservatives include, for example, methyl-p-hydroxybenzoate, ethyl-p-hydroxybenzoate, sorbic acid, phenol, cresol, and chlorocresol.

Adsorption inhibitors include, for example, human serum albumin, lecithin, dextran, ethylene oxide/propylene oxide copolymer, hydroxypropyl cellulose, methyl cellulose, polyoxyethylene hardened castor oil, and polyethylene glycol.

Sulfur-containing reducing agents include, for example, those containing sulfhydryl groups such as N-acetylcysteine, N-acetylhomocysteine, thioctic acid, thiodiglycol, thioethanol amine, thioglycerol, thiosorbitol, thioglycolic acid and salts thereof, sodium thiosulfate, glutathione, and thioalkanoic acids having one to seven carbon atoms.

Antioxidants include, for example, erythorbic acid, dibutylhydroxytoluene, butylhydroxyanisole, α-tocopherol, tocopherol acetate, L-ascorbic acid and salts thereof,

L-ascorbic acid palmitate, L-ascorbic acid stearate, sodium hydrogen sulfite, sodium sulfite, triamyl gallate, propyl gallate, and chelating agents such as disodium ethylenediamine tetraacetate (EDTA), sodium pyrophosphate, and sodium metaphosphate.

In an embodiment, the formulation of the present invention is the following: an antibody solution formulation of pH 6, which comprises:
a bispecific antibody at 20 to 180 mg/mL, wherein a first polypeptide and a third polypeptide form a pair, and a second polypeptide and a fourth polypeptide form a pair, wherein the first polypeptide comprises an H chain comprising the amino acid sequence of SEQ ID NO: 10, the second polypeptide comprises an H chain comprising the amino acid sequence of SEQ ID NO: 11, and the third polypeptide and the fourth polypeptide comprises a common L chain of SEQ ID NO: 12;
20 mM L-histidine/aspartate buffer;
0.5 mg/mL Poloxamer 188; and
150 mM L-arginine;
   or
   an antibody solution formulation of pH 6, which comprises:
the bispecific antibody Emicizumab (ACE910) at 20 to 180 mg/mL,
20 mM L-histidine/aspartate buffer;
0.5 mg/mL Poloxamer 188; and
150 mM L-arginine.

In another embodiment, the formulation of the present invention is the following: an antibody solution formulation of pH 6, which comprises:
a bispecific antibody at 20 to 180 mg/mL, wherein a first polypeptide and a third polypeptide form a pair, and a second polypeptide and a fourth polypeptide form a pair, wherein the first polypeptide comprises an H chain comprising the amino acid sequence of SEQ ID NO: 10, the second polypeptide comprises an H chain comprising the amino acid sequence of SEQ ID NO: 11, and the third polypeptide and the fourth polypeptide comprise a common L chain of SEQ ID NO: 12;
20 mM L-histidine/aspartate buffer;
0.05 mg/mL PS20; and
150 mM L-arginine;
   or
   an antibody solution formulation of pH 6, which comprises:
the bispecific antibody Emicizumab (ACE910) at 20 to 180 mg/mL,
20 mM L-histidine/aspartate buffer;
0.05 mg/mL PS20; and
150 mM L-arginine.

The antibody-containing formulations of the present invention can be administered to a patient via any appropriate route, for example, by bolus injection or continuous infusion for a certain period, intravenously, intramuscularly, or subcutaneously. Intravenous administration or subcutaneous administration is preferred.

The dosage of Emicizumab (ACE910) is, for example, 0.001 to 1000 mg/kg, and the interval of administration is at least one day or longer.

More specifically, for example, after administering Emicizumab (ACE910) at an initial dose of 1 mg/kg, Emicizumab (ACE910) can be administered at a continuous dose of 0.3 mg/kg once a week. Alternatively, for example, after administering Emicizumab (ACE910) at an initial dose of 3 mg/kg, Emicizumab (ACE910) can be administered at a continuous dose of 1 mg/kg once a week. In another example, after administering Emicizumab (ACE910) at an initial dose of 3 mg/kg, Emicizumab (ACE910) can be administered at a continuous dose of 3 mg/kg once a week.

Antibody-containing formulations of the present invention can be used for diseases that develop and/or progress due to the reduction or deficiency in the activity of FVIII and/or activated blood coagulation factor VIII (FVIIIa). For example, they can be used for hemophilia A, hemophilia A in which an inhibitor against FVIII/FVIIIa has appeared, acquired hemophilia A, von Willebrand's disease, without being particularly limited thereto.

Another embodiment of the present invention is a method for stabilizing an antibody in an antibody-containing solution formulation. Preferably, the method for stabilizing an antibody in an antibody-containing solution formulation comprises adding a histidine/aspartate buffer, Poloxamer 188, and arginine to the solution.

Another embodiment of the present invention is a method for reducing association (aggregate formation) of an antibody in an antibody-containing solution formulation. Preferably, the method for reducing association (aggregate formation) of an antibody in an antibody-containing solution formulation comprises adding a histidine/aspartate buffer, Poloxamer 188, and arginine to the solution.

Furthermore, the above-mentioned method for stabilizing an antibody and method for reducing association (aggregate formation) of an antibody comprise adding a histidine/aspartate buffer, Poloxamer 188, and arginine to the solution, and preferably the antibody concentration is 20 to 180 mg/mL, histidine/aspartate buffer concentration is 10 mM to 40 mM, Poloxamer 188 concentration is 0.2 to 1 mg/mL, arginine concentration is 100 mM to 300 mM, and pHis 4.5 to 6.5; or more preferably the antibody concentration is 20 to 180 mg/mL, histidine/aspartate buffer concentration is 20 mM, Poloxamer 188 concentration is 0.5 mg/mL, arginine concentration is 150 mM, and pH is 6.

Another embodiment of the present invention is a method for decreasing a component with charge heterogeneity in an antibody-containing formulation. Preferably, the method for decreasing a component with charge heterogeneity in an antibody-containing formulation comprises adding a histidine/aspartate buffer to the solution. More preferably, the method for decreasing a component with charge heterogeneity in an antibody-containing formulation comprises adding a histidine/aspartate buffer to the solution, where the histidine/aspartate buffer concentration is 10 mM to 40 mM, or at 20 mM.

In another embodiment of the present invention, the method for decreasing a component with charge heterogeneity in an antibody-containing formulation comprises adding a histidine/aspartate buffer, Poloxamer 188, and arginine to the solution. More preferably, the method for decreasing a component with charge heterogeneity in an antibody-containing formulation comprises adding a histidine/aspartate buffer, Poloxamer 188, and arginine to the solution, and preferably the antibody concentration is 20 to 180 mg/mL, histidine/aspartate buffer concentration is 10 mM to 40 mM, Poloxamer 188 concentration is 0.2 to 1 mg/mL, arginine concentration is 100 mM to 300 mM, and pH is 4.5 to 6.5, or more preferably the antibody concentration is 20 to 180 mg/mL, histidine/aspartate buffer concentration is 20 mM, Poloxamer 188 concentration is 0.5 mg/mL, arginine concentration is 150 mM, and pH is 6.

In the above-mentioned method for stabilizing an antibody, the method for reducing association (aggregate formation) of an antibody, and the method for decreasing a component with charge heterogeneity, the antibody is preferably a bispecific antibody and more preferably Emicizumab (ACE910).

As used herein, aspects referred to by the expression “comprising” include those referred to by the expression “essentially consisting of”, and those referred to by the expression “consisting of”.

Numerical values recited herein may vary within a certain range, for example, depending on the instruments or equipment, measurement conditions, and procedure used by those skilled in the art, and so long as they are within a range that allows the objective of the invention to be accomplished, they may encompass a deviation of approximately 10%, for example.

All patents and references explicitly cited herein are incorporated by reference into this specification in its entirety.

The present invention will be further illustrated by the Examples below, but it is not to be construed as being limited thereto.

### EXAMPLES

### Example 1

### Aggregate-Suppressing Effects of Histidine During Thermally Accelerated Storage of the Humanized IgG4 Antibody ACE910

### (1) Materials

ACE910 is a bispecific humanized IgG4 antibody that recognizes both blood coagulation factor IX and blood coagulation factor X, which is expected to prevent bleeding in hemophilia A by functionally substituting for activated blood coagulation factor VIII.

### (2) Test Samples

Liquid compositions of pH 6.0 containing ACE910 at 100 mg/mL, NaCl at 150 mmol/L, and any one the following buffers: Phosphate buffer at 20 mmol/L; Citrate buffer at 20 mmol/L; Acetate buffer at 20 mmol/L; and Histidine buffer at 20 mmol/L, were prepared. Glass vials were respectively filled with 5 to 15 μL of the compositions.

Humanized antibody-containing solution formulations thus prepared were left to stand in a thermo-regulated bath at 25 °C for eight weeks, and then used as test samples.

### (3) Methods for Measuring and Calculating the Amount of ACE910 Aggregates

The amounts of aggregates in the samples were measured by size exclusion chromatography (SEC) using a G3000SW_{XL} (Tosoh) column with phosphate buffer (50 mmol/L, pH7.0) containing sodium chloride at 300 mmol/L for the mobile phase at a flow rate of 0.5 mL/min.

Of the detected peaks, the peak with the largest area and height was determined to be the monomer, and the peaks detected earlier than the monomer were collectively referred to as aggregate peaks (high molecular weight species, HMWS).

The peak areas were calculated for all of the peaks, and the peak area ratio of the peak of interest was calculated using the following equation:

The peak area ratio of the peak of interest(%)=100×(the peak area of the peak of interest)/(the peak area of the peak of interest+the total peak area of the other peaks)

### (4) Results

The obtained results are shown in Table 1.

TABLE 1
Increase in aggregates (%) after storage at 25 °C

| | Δ HMW (%) | | |
|---|---|---|---|
| Formulation | 25 °C-2 W | 25 °C-4 W | 25 °C-8 W |
| Phosphate | 0.20 | 0.27 | 0.41 |
| Citrate | 0.14 | 0.21 | 0.32 |
| Acetate | 0.24 | 0.38 | 0.63 |
| Histidine | 0.08 | 0.13 | 0.22 |

As is clear from Table 1, when supplemented with histidine at 20 mmol/L, the sample showed a high aggregate-suppressing effect after thermal acceleration at 25 °C for eight weeks.

### Example 2

### Aggregate-Suppressing Effects of the Salt Concentration and Arginine During Thermally Accelerated Storage and Freeze-Thawing of the Humanized IgG4 Antibody ACE910

### (1) Materials

The antibody described in Example 1 was used.

### (2) Test Samples

Liquid compositions of pH 6.0 containing ACE910 at 100 mg/mL, Histidine at 20 mmol/L, and any one of the following additives: NaCl at 50 mmol/L; NaCl at 75 mmol/L; NaCl at 150 mmol/L; and Arginine at 150 mmol/L, were prepared. Glass vials were respectively filled with 5 to 15 μL of the compositions.

Humanized antibody-containing solution formulations thus prepared were left to stand in a thermo-regulated bath at 25 °C for eight weeks, or were subjected to ten cycles of freeze-thawing (F/T) (5 °C/−20° °C), and then used as test samples.

### (3) Methods for Measuring and Calculating the Amount of ACE910 Aggregates

The methods were performed as described in Example 1.

### (4) Results

The obtained results are shown in Table 2.

TABLE 2
Increase in aggregates (%) after storage
at 25 °C and after freeze-thawing

| | | Δ HMW (%) | | | | |
|---|---|---|---|---|---|---|
| | | | | 25 | 25 | 25 |
| Formulation | | 5 F/T | 10 F/T | °C-2 W | °C-4 W | °C-8 W |
| 50 | mM NaCl | 1.00 | 2.07 | 0.09 | 0.16 | 0.26 |
| 75 | mM NaCl | 0.62 | 1.43 | 0.07 | 0.13 | 0.23 |
| 150 | mM NaCl | 0.11 | 0.28 | 0.08 | 0.13 | 0.22 |
| 150 | mM Arg | 0.03 | 0.05 | 0.03 | 0.06 | 0.11 |

As is clear from Table 2, when supplemented with arginine at 150 mmol/L, the samples showed a high aggregate-suppressing effect after the thermal acceleration test at 25 °C for eight weeks and the freeze-thawing.

### Example 3

### Aggregate-Suppressing Effects of Aspartic Acid During Freeze-Thawing of the Humanized IgG4 Antibody ACE910

### (1) Materials

The antibody described in Example 1 was used.

### (2) Test Samples

Liquid compositions of pH 6.0 containing ACE910 at 100 mg/mL, Histidine at 20 mmol/L, and NaCl at 150 mmol/L or sodium L-Aspartic acid at 150 mmol/L as the counter ion, were prepared. Glass vials were respectively filled with 5 to 15 μL of the compositions. Humanized antibody-containing solution formulations thus prepared were subjected to ten cycles of freeze-thawing (5 °C/−20 °C), and then used as test samples.

### (3) Methods for Measuring and Calculating the Amount of ACE910 Aggregates

The methods were performed as described in Example 1.

### (4) Results

The obtained results are shown in Table 3.

TABLE 31
Increase in aggregates (%) after freeze-thawing

Δ HMW (%)

| Formulation | 5 F/T | 10 F/T |
|---|---|---|
| NaCl | 0.11 | 0.28 |
| Na Aspartic acid | 0.05 | 0.13 |

As is clear from Table 3, when supplemented with aspartic acid, the samples showed a high aggregate-suppressing effect after the freeze-thawing.

### Example 4

Effects of Suppressing Aggregates and Components with Charge Heterogeneity by pH During Thermally Accelerated Storage of the Humanized IgG4 Antibody ACE910

### (1) Materials

The antibody described in Example 1 was used.

### (2) Test Samples

Liquid compositions containing ACE910 at 100 mg/mL, Histidine-Aspartic acid at 20 mmol/L, and Arginine-Aspartic acid at 150 mmol/L, and having pH of 4.5, 5.0, 5.5, 6.0, 6.5, 7.0, or 7.5 were prepared. Glass vials were respectively filled with 5 to 15 μL of the compositions. Humanized antibody-containing solution formulations thus prepared were left to stand in a thermo-regulated bath at 25 °C for eight weeks, and then used as test samples.

### (3) Methods for Measuring and Calculating the Amount of ACE910 Aggregates

The methods were performed as described in Example 1.

(4) Methods for Measuring and Calculating ACE910 Components with Charge Heterogeneity

The amount of components with charge heterogeneity in a sample was measured by ion exchange chromatography (IEC) through a BioPro QA-F column (YMC) using Tris-HCl buffer (20 mmol/L, pH 7.8) as mobile phase A and Tris-HCl buffer (20 mmol/L, pH7.8) containing sodium chloride (500 mmol/L) as mobile phase B, at a flow rate of 0.5 mL/min.

Of the detected peaks, the peak with the largest area and height was determined to be the Main peak, and the peaks detected later than the Main peak were collectively referred to as the Acidic peak.

The peak area was calculated for all of the peaks, and the peak area ratio of the peak of interest was calculated using the following equation:

The peak area ratio of the peak of interest(%)=100×(the peak area of the peak of interest)/(the peak area of the peak of interest+the total peak area of the other peaks)

### (5) Results

The obtained results are shown in Table 4.

TABLE 4
Increase in aggregates (%) and increase in Acidic peak-1 (%) after storage at 25 °C

| | Δ HMW (%) | | | Δ Acidic-1 (%) | | |
|---|---|---|---|---|---|---|
| Formulation | 25 °C-2 W | 25 °C-4 W | 25 °C-8 W | 25 °C-2 W | 25 °C-4 W | 25 °C-8 W |
| pH 4.5 | 0.05 | 0.10 | 0.24 | 0.21 | 2.43 | 3.41 |
| pH 5.0 | 0.09 | 0.11 | 0.20 | 0.46 | 0.86 | 2.57 |
| pH 5.5 | 0.07 | 0.08 | 0.15 | 0.36 | 0.68 | 2.10 |
| pH 6.0 | 0.07 | 0.11 | 0.17 | 0.38 | 0.47 | 3.23 |
| pH 6.5 | 0.09 | 0.14 | 0.25 | 0.24 | 1.17 | 3.90 |
| pH 7.0 | 0.13 | 0.17 | 0.30 | 1.25 | 2.44 | 5.39 |
| pH 7.5 | 0.18 | 0.32 | 0.79 | 3.09 | 5.01 | 9.49 |

As is clear from Table 4, the samples at pH 4.5 to 6.5, and in particular at pH 5.5 and pH 6.0, showed a high effect of suppressing aggregates and components with charge heterogeneity after storage at 25 °C.

### Example 5

Effects of Suppressing Aggregates and Components with Charge Heterogeneity by the Histidine Concentration During Thermally Accelerated Storage of the Humanized IgG4 Antibody ACE910

### (1) Materials

The antibody described in Example 1 was used.

### (2) Test Samples

Liquid compositions of pH 6.0 containing ACE910 at 100 mg/mL, Arginine at 150 mmol/L, and Histidine-aspartic acid at 5 mmol/L, 10 mmol/L, 20 mmol/L, or 40 mmol/L were prepared. Glass vials were respectively filled with 5 to 15 μL of the compositions. Humanized antibody-containing solution formulations thus prepared were left to stand in a thermo-regulated bath at 25 °C for eight weeks, and then used as test samples.

### (3) Methods for Determining and Calculating the Amount of ACE910 Aggregates

The methods were performed as described in Example 1.

(4) Methods for Measuring and Calculating ACE910 Components with Charge Heterogeneity

The methods were performed as described in Example 4.

### (5) Results

The obtained results are shown in Table 5.

TABLE 5
Increase in aggregates (%) and increase in Acidic peak-1 (%) after storage at 25 °C

| Histidine | Δ HMW (%) | | | Δ Acidic-1 (%) | | |
|---|---|---|---|---|---|---|
| concentration | 25 °C-2 W | 25 °C-4 W | 25 °C-8 W | 25 °C-2 W | 25 °C-4 W | 25 °C-8 W |
| 5 mmol/L | 0.04 | 0.09 | 0.17 | 0.87 | 3.01 | 9.19 |
| 10 mmol/L | 0.02 | 0.05 | 0.14 | 0.39 | 1.50 | 7.09 |
| 20 mmol/L | 0.01 | 0.04 | 0.12 | 0.06 | 1.04 | 7.11 |
| 40 mmol/L | 0.01 | 0.02 | 0.07 | 0.52 | 1.13 | 6.74 |

As is clear from Table 5, the samples containing 10 mmol/L or more of Histidine-aspartic acid showed a high effect of suppressing aggregates and components with charge heterogeneity after storage at 25 °C.

### Example 6

### Aggregate-Suppressing Effects of the Arginine Concentration During Freeze-Thawing, Thermally Accelerated Storage, and Cryopreservation of the Humanized IgG4 Antibody ACE910

### (1) Materials

The antibody described in Example 1 was used.

### (2) Test Samples

Liquid compositions of pH 6.0 containing ACE910 at 100 mg/mL, Histidine-aspartic acid at 20 mmol/L, and Arginine at 75 mmol/L, 100 mmol/L, 150 mmol/L, 200 mmol/L, or 300 mmol/L were prepared. Glass vials were respectively filled with 5 to 15 μL of the compositions.

Humanized antibody-containing solution formulations thus prepared were subjected to ten cycles of freeze-thawing (5 °C/−20 °C), or left to stand in a thermo-regulated bath at 25 °C for eight weeks or at −20 °C for six months, and then used as test samples.

### (3) Methods for Measuring and Calculating the Amount of ACE910 Aggregates

The methods were performed as described in Example 1.

### (4) Results

The obtained results are shown in Table 6.

TABLE 6
Increase in aggregates (%) after freeze-thawing, after storage at 25 °C, and after storage at −20 °C

| Arginine | Δ HMW (%) | | | | | | |
|---|---|---|---|---|---|---|---|
| concentration | 5 F/T | 10 F/T | 25 °C-2 W | 25 °C-4 W | 25 °C-8 W | −20 °C-3 M | −20 °C-6 M |
| 75 mmol/L | 0.10 | 0.10 | 0.02 | 0.06 | 0.17 | 0.19 | 0.53 |
| 100 mmol/L | 0.04 | 0.04 | 0.01 | 0.04 | 0.12 | 0.08 | 0.02 |
| 150 mmol/L | 0.01 | 0.01 | −0.01 | 0.01 | 0.04 | 0.00 | 0.00 |
| 200 mmol/L | 0.01 | 0.01 | −0.01 | 0.00 | 0.02 | 0.00 | −0.01 |
| 300 mmol/L | 0.00 | 0 00 | −0.02 | −0.02 | 0.00 | 0.00 | −0.01 |

As is clear from Table 6, the samples containing Arginine at 100 mmol/L or more showed high aggregate-suppressing effects after freeze-thawing, after storage at 25 °C, and after storage at −20 °C.

### Example 7

Effects of suppressing insoluble foreign substances and insoluble microparticles by Poloxamer 188 during storage at 5 °C of the humanized IgG4 antibody ACE910

### (1) Materials

The antibody described in Example 1 was used.

### (2) Test Samples

Liquid compositions of pH 6.0 containing ACE910 at 80 mg/mL, Histidine-Aspartic acid at 20 mmol/L, Arginine at 150 mmol/L, and any one of the following additives: Poloxamer 188 at 0 mg/mL; Poloxamer 188 at 0.2 mg/mL; Poloxamer 188 at 0.5 mg/mL; Poloxamer 188 at 1.0 mg/mL; Polysorbate20 at 0.05 mg/mL; and Polysorbate20 at 1.0 mg/mL, were prepared. Glass vials were respectively filled with 1.0 mL of the compositions.

Humanized antibody-containing solution formulations thus prepared were left to stand in a refrigerator at 5 °C for five months, and then used as test samples.

### (3) Method for Observing Insoluble Foreign Substances

The presence of insoluble foreign substances was assessed by placing the sample on a sample platform of a visual examination stand for vials, rotating the sample platform, and observing the vial.

### (4) Method for Measuring Insoluble Microparticles

The number of insoluble microparticles in a solution was determined using a liquid microparticle counter (Hach Ultra Analytics, Model 9703).

### (5) Results

The obtained results are shown in Table 7.

TABLE 7
Number of insoluble microparticles (microparticle/mL) and detection
rate of insoluble foreign substances (%) after storage at 5 °C

| | | | 0.2 mg/mL | | | | 0.5 mg/mL | | | | 1.0 mg/mL | | | |
|---|---|---|---|---|---|---|---|---|---|---|---|---|---|---|
| PX188 | | | Initial | 1 M | 3 M | 5 M | Initial | 1 M | 3 M | 5 M | Initial | 1 M | 3 M | 5 M |
| Number of insoluble | (Micropar- | ≥5 μm | 31 | — | 61 | 188 | 9 | — | 18 | 27 | 7 | — | 7 | 43 |
| microparticles | ticles/mL) | ≥10 μm | 5 | — | 4 | 15 | 5 | — | 3 | 2 | 5 | — | 3 | 5 |
| in solution | | ≥25 μm | 1 | — | 1 | 1 | 1 | — | 0 | 0 | 0 | — | 1 | 1 |

| | | | | | | | | | | | | |
|---|---|---|---|---|---|---|---|---|---|---|---|---|
| Detection rate of insoluble foreign substances | 0/5 | 0/5 | 0/5 | 0/5 | 0/5 | 0/5 | 0/5 | 0/5 | 0/5 | 0/5 | 0/5 | 0/5 |
| (number of vials containing foreign | | | | | | | | | | | | |
| substances/number of examined vials) | | | | | | | | | | | | |

| | | | 0.5 mg/mL | | | | 1.0 mg/mL | | | |
|---|---|---|---|---|---|---|---|---|---|---|
| PS20 | | | Initial | 1 M | 3 M | 5 M | Initial | 1 M | 3 M | 5 M |
| Number of insoluble | (Micropar- | ≥5 μm | 13 | — | 36 | 89 | 24 | — | 238 | 411 |
| microparticles | ticles/mL) | ≥10 μm | 5 | — | 3 | 10 | 9 | — | 49 | 66 |
| in solution | | ≥25 μm | 0 | — | 0 | 0 | 1 | — | 3 | 2 |

| | | | | | | | | |
|---|---|---|---|---|---|---|---|---|
| Detection rate of insoluble foreign substances | 0/5 | 0/5 | 0/5 | 0/5 | 0/5 | 0/5 | 5/5 | 5/5 |
| (number of vials containing foreign | | | | | | | | |
| substances/number of examined vials) | | | | | | | | |

As is clear from Table 7, the samples containing PS20 at 0.05 mg/mL and the samples containing Poloxamer 188 at 0.2 mg/mL or more showed a high effect of suppressing the formation of insoluble microparticles and insoluble foreign substances after storage at 5 °C.

### Example 8

Effects of suppressing insoluble foreign substances and insoluble microparticles by Poloxamer 188 during a shaking stress and freeze-thaw storage of the humanized IgG4 antibody ACE910

### (1) Materials

The antibody described in Example 1 was used.

(2) Test samples

Liquid compositions of pH 6.0 containing ACE910 at 150 mg/mL, Histidine-Aspartic acid at 20 mmol/L, Arginine-Aspartic acid at 150 mmol/L, and any one of the following additives: Poloxamer 188 at 0 mg/mL; Poloxamer 188 at 0.2 mg/mL; Poloxamer 188 at 0.5 mg/mL; and Poloxamer 188 at 0.8 mg/mL, were prepared. Glass vials were respectively filled with 0.9 mL of the compositions.

Humanized antibody-containing solution formulations thus prepared were subjected to shaking for 24 hours at a speed of 200 strokes/min using a shaker at room temperature, or ten cycles of freeze-thawing (5 °C/−20 °C), and then used as test samples.

### (3) Method for Observing Insoluble Substances

The method was performed as described in Example 7.

### (4) Method for Measuring Insoluble Microparticles

The method was performed as described in Example 7.

### (5) Results

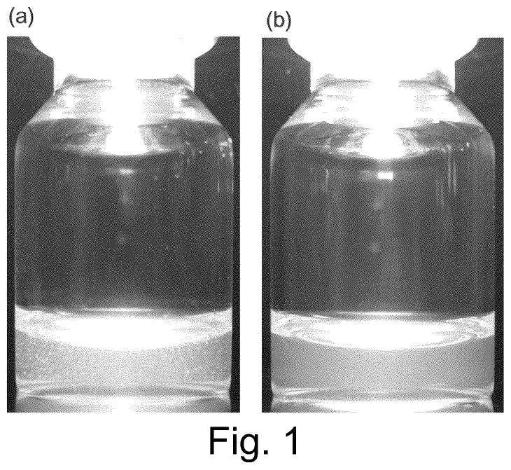

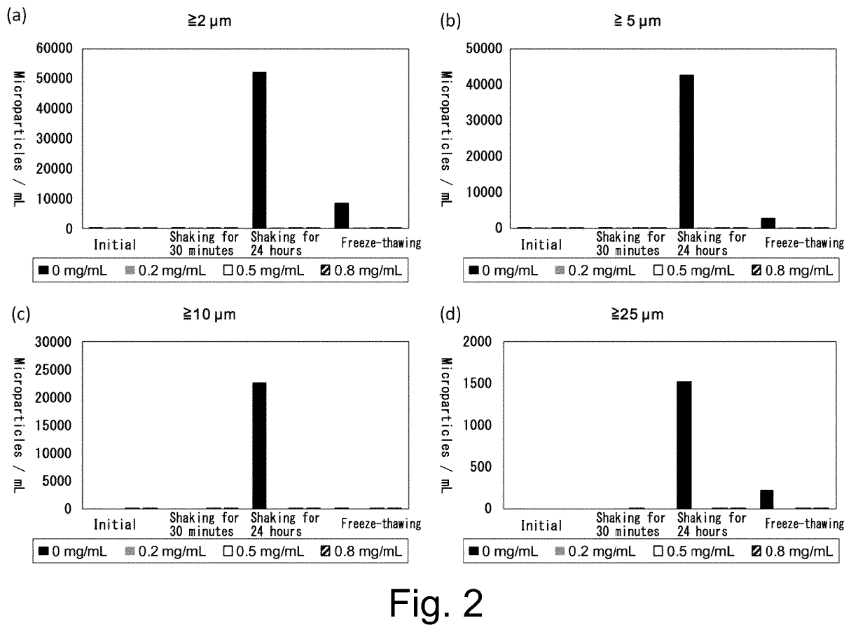

The obtained results are shown in Table 8 and FIGS. 1 and 2.

TABLE 8
Detection rate (%) for insoluble foreign substances
after shaking and freeze-thaw storage

| | Detection rate for insoluble foreign substances | | | |
|---|---|---|---|---|
| | (number of vials containing foreign | | | |
| Poloxamer | substances/number of examined vials) | | | |
| 188 | | Shaking for | Shaking for | Freeze- |
| concentration | Initial | 30 minutes | 24 hours | thawing |
| 0 mg/mL | 0% (0/10) | 50% (5/10) | 100% (10/10) | 0% (0/10) |
| 0.2 mg/mL | 0% (0/10) | 0% (0/10) | 0% (0/10) | 0% (0/10) |
| 0.5 mg/mL | 0% (0/10) | 0% (0/10) | 0% (0/10) | 0% (0/10) |
| 0.8 mg/mL | 0% (0/10) | 0% (0/10) | 0% (0/10) | 0% (0/10) |

As is clear from Table 8 and FIGS. 1 and 2, the samples containing Poloxamer 188 at 0.2 mg/mL or more showed a high effect of suppressing the formation of insoluble microparticles and insoluble foreign substances after being subjected to shaking stress and freeze-thaw storage.

### Example 9

### Effects of the Concentration of Humanized IgG4 Antibody ACE910 on the Stability During Thermally Accelerated Storage and Freeze-Thaw Storage

### (1) Materials

The antibody described in Example 1 was used.

### (2) Test Samples

Liquid compositions of pH 6.0 containing Histidine-Aspartic acid at 20 mmol/L, Arginine-Aspartic acid at 150 mmol/L, Poloxamer 188 at 0.5 mg/mL, and ACE910 at 20 mg/mL, 30 mg/mL, 40 mg/mL, 120 mg/mL, 150 mg/mL, or 180 mg/mL were prepared. Glass vials were respectively filled with 0.65 mL of the compositions.

Humanized antibody-containing solution formulations thus prepared were left to stand in a thermo-regulated bath at 40 °C for eight weeks, or subjected to five or ten cycles of freeze-thawing (25 °C/−20 °C), and then used as test samples.

### (3) Methods for Measuring and Calculating the Amount of ACE910 Aggregates

The methods were performed as described in Example 1.

(4) Methods for Measuring and Calculating ACE910 Components with Charge Heterogeneity

The amount of components with charge heterogeneity in a sample was measured by anion exchange chromatography (AIEC) through a TSKgel Q-STAT column (Waters) using Tris-HCl buffer (50 mmol/L, pH 8.0) as mobile phase A and Tris-HCl buffer (50 mmol/L, pH8.0) containing sodium chloride (200 mmol/L) as mobile phase B at a flow rate of 0.5 mL/min.

Of the detected peaks, the peak with the largest area and height was determined to be the Main peak, and the peaks detected earlier than the Main peak were collectively referred to as the Basic peaks and the peaks detected later than the Main peak were collectively referred to as the Acidic peaks.

Furthermore, the amount of components with charge heterogeneity was measured by cation exchange chromatography (CIEC) through a ProPac WCX-10G column (Thermo Scientific) using a buffer containing Tris at 9.6 mmol/L, piperazine at 6.0 mmol/L, and imidazole at 11.0 mmol/L (pH 6.0) as mobile phase A and a buffer containing Tris at 9.6 mmol/L, piperazine at 6.0 mmol/L, imidazole at 11.0 mmol/L, and NaCl at 100 mmol/L (pH 10.1) as mobile phase B at a flow rate of 0.5 mL/min.

Of the detected peaks, the peak with the largest area and height was determined to be the BiAb peak, the peaks detected earlier than the BiAb peak were collectively referred to as the Pre peaks and the peaks detected later than the BiAb peak were collectively referred to as the Post peaks.

The peak area was calculated for all of the peaks, and the peak area ratio of the peak of interest was calculated using the following equation:

The peak area ratio of the peak of interest(%)=100×(the peak area of the peak of interest)/(the peak area of the peak of interest+the total peak area of the other peaks)

### (5) Results

The obtained results are shown in Table 9. “SE”, “AE”, and “CE” respectively indicate the results from size exclusion chromatography, anion exchange chromatography, and cation exchange chromatography.

TABLE 9
Amounts of aggregates (%) and components with charge heterogeneity
(%) after storage at 40 °C and after freeze-thawing

| ACE910 conc. (mg/mL) | | 20 | 30 | 40 | 120 | 150 | 180 |
|---|---|---|---|---|---|---|---|
| SE-HPLC | Initial | 0.10 | 0.11 | 0.11 | 0.13 | 0.14 | 0.14 |
| % HMWS | 2 weeks 40 °C | 0.14 | 0.15 | 0.16 | 0.26 | 0.28 | 0.31 |
| | 4 weeks 40 °C | 0.16 | 0.17 | 0.20 | 0.34 | 0.38 | 0.44 |
| | 5 cycles F/T | 0.11 | 0.11 | 0.12 | 0.13 | 0.14 | 0.14 |
| | 10 cycles F/T | 0.11 | 0.11 | 0.11 | 0.13 | 0.13 | 0.14 |
| SE-HPLC | Initial | 99.90 | 99.89 | 99.89 | 99.87 | 99.86 | 99.86 |
| % Monomer | 2 weeks 40 °C | 99.86 | 99.85 | 99.84 | 99.74 | 99.72 | 99.69 |
| | 4 weeks 40 °C | 99.84 | 99.83 | 99.80 | 99.66 | 99.62 | 99.56 |
| | 5 cycles F/T | 99.89 | 99.89 | 99.88 | 99.87 | 99.86 | 99.86 |
| | 10 cycles F/T | 99.89 | 99.89 | 99.89 | 99.87 | 99.87 | 99.86 |
| AE-HPLC | Initial | 16.1 | 16.2 | 16.1 | 16.3 | 16.0 | 16.3 |
| % Basic | 2 weeks 40 °C | 14.3 | 14.6 | 14.4 | 14.5 | 14.5 | 14.5 |
| region | 4 weeks 40 °C | 12.7 | 12.8 | 12.8 | 12.8 | 12.7 | 12.7 |
| | 5 cycles F/T | 16.1 | 16.2 | 16.4 | 16.5 | 16.5 | 16.5 |
| | 10 cycles F/T | 15.9 | 16.0 | 16.2 | 16.4 | 16.5 | 16.4 |
| AE-HPLC | Initial | 71.7 | 71.6 | 71.8 | 71.5 | 72.1 | 71.6 |
| % Main | 2 weeks 40 °C | 65.0 | 64.6 | 64.7 | 64.5 | 63.9 | 64.4 |
| | 4 weeks 40 °C | 58.3 | 57.8 | 57.8 | 57.2 | 57.3 | 57.5 |
| | 5 cycles F/T | 72.3 | 72.2 | 72.0 | 71.6 | 71.6 | 71.6 |
| | 10 cycles F/T | 72.7 | 72.4 | 72.1 | 71.5 | 71.6 | 71.7 |
| AE-HPLC | Initial | 12.1 | 12.2 | 12.1 | 12.2 | 11.9 | 12.1 |
| % Acdic | 2 weeks 40 °C | 20.7 | 20.8 | 20.9 | 20.9 | 21.6 | 21.1 |
| region | 4 weeks 40 °C | 29.0 | 29.4 | 29.4 | 30.0 | 30.0 | 29.8 |
| | 5 cycles F/T | 11.6 | 11.6 | 11.6 | 11.9 | 11.9 | 12.0 |
| | 10 cycles F/T | 11.5 | 11.6 | 11.7 | 12.1 | 12.0 | 11.9 |
| CE-HPLC | Initial | 3.2 | 3.2 | 3.3 | 3.3 | 3.3 | 3.3 |
| % Pre- | 2 weeks 40 °C | 3.6 | 3.8 | 3.8 | 3.7 | 3.7 | 3.7 |
| peaks | 4 weeks 40 °C | 4.3 | 4.3 | 4.3 | 4.3 | 4.2 | 4.3 |
| | 5 cycles F/T | 3.4 | 3.0 | 3.2 | 3.1 | 3.0 | 3.1 |
| | 10 cycles F/T | 3.1 | 3.2 | 2.8 | 3.1 | 3.1 | 3.1 |
| CE-HPLC | Initial | 96.8 | 96.8 | 96.7 | 96.6 | 96.6 | 96.6 |
| % BiAb | 2 weeks 40 °C | 96.4 | 96.2 | 96.3 | 96.3 | 96.3 | 96.2 |
| | 4 weeks 40 °C | 95.6 | 95.7 | 95.6 | 95.7 | 95.8 | 95.8 |
| | 5 cycles F/T | 96.6 | 97.0 | 96.8 | 96.9 | 97.0 | 96.9 |
| | 10 cycles F/T | 96.8 | 96.7 | 97.2 | 96.8 | 96.9 | 96.9 |
| CE-HPLC | Initial | 0.0 | 0.0 | 0.0 | 0.1 | 0.1 | 0.1 |
| % Post- | 2 weeks 40 °C | 0.0 | 0.0 | 0.0 | 0.0 | 0.0 | 0.0 |
| peaks | 4 weeks 40 °C | 0.0 | 0.0 | 0.0 | 0.0 | 0.0 | 0.0 |
| | 5 cycles F/T | 0.0 | 0.0 | 0.0 | 0.0 | 0.0 | 0.0 |
| | 10 cycles F/T | 0.0 | 0.0 | 0.0 | 0.0 | 0.0 | 0.0 |

As is clear from Table 9, comparing the samples containing ACE910 at 20 mg/mL to 180 mg/mL, it was shown that the samples had an equivalent, sufficient stability after storage at 40 °C and after freeze-thawing.

### INDUSTRIAL APPLICABILITY

Compared to conventional formulations, the antibody solution formulations of the present invention have a superior stability in the solution state, and show suppressed aggregate formation of proteins such as the antibody molecules after storage at low, ambient, and high temperatures, and after freeze-thawing. The antibody solution formulations of the present invention in which deterioration reactions are difficult to occur can be used, for example, to treat hemophilia A by subcutaneous administration.

1. An antibody solution formulation of pH 4.5 to 6.5, which comprises:
a bispecific antibody at 20 to 180 mg/mL, wherein a first polypeptide and a third polypeptide form a pair, and a second polypeptide and a fourth polypeptide form a pair, wherein the first polypeptide comprises an H chain comprising the amino acid sequences of H-chain CDRs 1, 2, and 3 of SEQ ID NOs: 1, 2, and 3 (H-chain CDRs of Q499), respectively; the second polypeptide comprises an H chain comprising the amino acid sequences of H-chain CDRs 1, 2, and 3 of SEQ ID NOs: 4, 5, and 6 (H-chain CDRs of J327), respectively; and the third polypeptide and the fourth polypeptide comprise a common L chain comprising the amino acid sequences of L-chain CDRs 1, 2, and 3 of SEQ ID NOs: 7, 8, and 9 (L-chain CDRs of L404), respectively;
10 mM to 40 mM histidine/aspartate buffer;
0.2 to 1 mg/mL Poloxamer 188; and
100 mM to 300 mM arginine.

2. The antibody solution formulation of claim 1, wherein in the bispecific antibody, the first polypeptide and the third polypeptide form a pair, and the second polypeptide and the fourth polypeptide form a pair, wherein the first polypeptide comprises an H chain comprising the amino acid sequence of SEQ ID NO: 10; the second polypeptide comprises an H chain comprising the amino acid sequence of SEQ ID NO: 11, and the third polypeptide and the fourth polypeptide comprise a common L chain of SEQ ID NO: 12.

3. The antibody solution formulation of claim 1 or 2, wherein the concentration of Poloxamer 188 is 0.5 mg/mL.

4. The antibody solution formulation of any one of claims 1 to 3, wherein said pH is 6.

5. The antibody solution formulation of any one of claims 1 to 4, wherein the concentration of histidine/aspartate buffer is 20 mM.

6. The antibody solution formulation of any one of claims 1 to 5, wherein the concentration of arginine is 150 mM.

7. The antibody solution formulation of any one of claims 1 to 6, which does not substantially contain a chloride ion or an acetate ion.

8. An antibody solution formulation of pH 6, which comprises:
a bispecific antibody at 20 to 180 mg/mL, wherein a first polypeptide and a third polypeptide form a pair, and a second polypeptide and a fourth polypeptide form a pair, wherein the first polypeptide comprises an H chain comprising the amino acid sequence of SEQ ID NO: 10; the second polypeptide comprises an H chain comprising the amino acid sequence of SEQ ID NO: 11; and the third polypeptide and the fourth polypeptide comprise a common L chain of SEQ ID NO: 12;
20 mM L-histidine/aspartate buffer;
0.5 mg/mL Poloxamer 188; and
150 mM L-arginine.

9. The antibody solution formulation of any one of claims 1 to 8, for use in subcutaneous administration.

10. The antibody solution formulation of any one of claims 1 to 9, for use in the treatment of hemophilia A.

11. A method for stabilizing an antibody in an antibody-containing solution formulation, which comprises adding a histidine/aspartate buffer, Poloxamer 188, and arginine to the solution, whereby the concentration of the histidine/aspartate buffer is 10 mM to 40 mM, the concentration of Poloxamer 188 is 0.2 to 1 mg/mL, and the concentration of arginine is 100 mM to 300 mM.

12. A method for suppressing the association (aggregate formation) of an antibody in an antibody-containing solution formulation, which comprises adding a histidine/aspartate buffer, Poloxamer 188, and arginine to the solution, whereby the concentration of the histidine/aspartate buffer is 10 mM to 40 mM, the concentration of Poloxamer 188 is 0.2 to 1 mg/mL, and the concentration of arginine is 100 mM to 300 mM.

13. A method for suppressing a component with charge heterogeneity in an antibody-containing formulation, which comprises adding a histidine/aspartate buffer to the solution, wherein the concentration of histidine/aspartate buffer is 10 mM to 40 mM.


SEQUENCE LISTING (15 sequences)

SEQ ID NO: 1 - protein, 5 aa, synthetic construct
  REGION 1..5: note = Heavy chain variable region CDR1
YYDIQ

SEQ ID NO: 2 - protein, 17 aa, synthetic construct
  REGION 1..17: note = Heavy chain variable region CDR2
SISPSGQSTYYRREVKG

SEQ ID NO: 3 - protein, 14 aa, synthetic construct
  REGION 1..14: note = Heavy chain variable region CDR3
RTGREYGGGWYFDY

SEQ ID NO: 4 - protein, 5 aa, synthetic construct
  REGION 1..5: note = Heavy chain variable region CDR1
DNNMD

SEQ ID NO: 5 - protein, 17 aa, synthetic construct
  REGION 1..17: note = Heavy chain variable region CDR2
DINTRSGGSIYNEEFQD

SEQ ID NO: 6 - protein, 10 aa, synthetic construct
  REGION 1..10: note = Heavy chain variable region CDR3
RKSYGYYLDE

SEQ ID NO: 7 - protein, 11 aa, synthetic construct
  REGION 1..11: note = Light chain variable region CDR1
KASRNIERQLA

SEQ ID NO: 8 - protein, 7 aa, synthetic construct
  REGION 1..7: note = Light chain variable region CDR2
QASRKES

SEQ ID NO: 9 - protein, 9 aa, synthetic construct
  REGION 1..9: note = Light chain variable region CDR3
QQYSDPPLT

SEQ ID NO: 10 - protein, 448 aa, synthetic construct
  REGION 1..448: note = Heavy chain
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 11 - protein, 444 aa, synthetic construct
  REGION 1..444: note = Heavy chain
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 12 - protein, 214 aa, synthetic construct
  REGION 1..214: note = Light chain
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 123 aa, synthetic construct
  REGION 1..123: note = Heavy chain variable region
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSS

SEQ ID NO: 14 - protein, 119 aa, synthetic construct
  REGION 1..119: note = Heavy chain variable region
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSS

SEQ ID NO: 15 - protein, 107 aa, synthetic construct
  REGION 1..107: note = Light chain variable region
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIK